(19) United States
(12) Patent Application Publication
(10) Pub. No.: US 2024/0017123 A1
(43) Pub. Date: Jan. 18, 2024

(54) EMOTION-LED WORKOUT GENERATOR

(71) Applicant: WATTCH FITNESS INC., Vancouver (CA)

(72) Inventors: Thomas Waller, North Vancouver (CA); J. Travis Stevenson, North Vancouver (CA)

(21) Appl. No.: 18/256,403

(22) PCT Filed: Dec. 7, 2021
(86) PCT No.: PCT/CA2021/051746

§ 371 Date: Jun. 7, 2023

Related U.S. Application Data
(60) Provisional application No. 63/186,036, filed on May 7, 2021.
(60) Provisional application No. 63/122,084, filed on Dec. 7, 2020.

(51) Int. Cl. A63B 24/00; A63B 71/06

(52) U.S. Cl. CPC A63B 24/0059; A63B 24/0062; A63B 24/0087; A63B 71/0622; A63B 2024/009

(57) ABSTRACT

Traditional workouts are sometimes harder to tolerate unless supplemented with entertainment content, even when the workouts are course-led, instructor-led and beat-led. By analyzing the emotional topography of an entertainment source, an emotion-led workout profile is created that is synchronized with non-quantitative elements of the source. Biometric responses may be included in the analysis. An exercise machine runs the emotion-led workout profile in synchronization with the entertainment. Controlling the machine to provide a sensory texture layer results in a more immersive experience. Potential benefits are the use of a single screen instead of two, an increase in actual effort compared to perceived effort, and increased enjoyment of a workout.

### TECHNICAL FIELD

This application is in the field of personal fitness. In particular, it relates to the control of workout machines.

### BACKGROUND

The global online and virtual fitness market is currently forecast to grow at 33% year-on-year. Through a wide variety of options, exercisers or ‘users’ are able to access on-demand or live workout content using smart TVs, smartphones, laptops, desktops, tablets, and digital displays built in to or accompanying exercise equipment such as stationary bikes, roller machines for regular bikes, treadmills, rowing machines and weight systems. Workout content provides direction through audio, verbal and/or visual cues and is often complemented by a graphical user interface with virtual courses and worlds, gamified environments with avatars, real or simulated videos of terrain, events or instructors, and is based on a predetermined sequence of exercise tasks.

Traditional workouts typically follow a variable effort profile that is linear, directly proportional or literal based on predetermined workout objectives, general exercise science principles and/or topographical terrain features. Workouts are often designed for exercise equipment such as bikes and treadmills, which have the functionality to vary the resistance through friction-based or magnetic braking, slope gradient or speed. Workouts are often published for the user to see resistance profiles or terrain in advance so they may select workout content they are interested in exercising to. During a workout, users are prompted to adjust resistance, slope, cadence or speed to match the workout design through audio, verbal and/or visual cues. The training effect that follows results in fatigue and, with appropriate recovery, the exerciser will become fitter and stronger over time.

The state of the art, online and virtual workout environments are increasingly enhanced by software designed to dynamically control the resistance, slope, speed and other aspects of connected fitness equipment in addition to providing workout recommendation, feedback or instruction, to control fidelity, to monitor performance and to encourage user response to traditional structured workouts.

There are three categories of traditional workouts: instructor led, course led, and beat led. Instructor-led workouts are structured, class-style environments with literal and regular direction from an instructor who provides specific commands for the user to adjust resistance, speed, load, technique etc. to encourage an applied effort. Often a curated music playlist accompanies the workout and users may choose or prefer a particular workout or instructor based on such things as instructor personality (e.g., humorous or inspiring) and musical genre (e.g., '90s Hip Hop or '80s Rock Anthems) in addition to the desired duration and effort profile. Instructor-led workouts are administered in person or remotely using online and virtual platforms that support real-time, live and pre-recorded classes and may be accompanied by one-way or two-way video or instructional content.

Course-led workouts allow users to follow programmed resistance profiles that are derived from known exercise science (e.g., intervals, VO₂ Max, Tempo) or literal representation of topographical terrain (e.g., mountain climb) where there tends to be a direct relationship between the terrain and required effort. Users progress through the workout profile or course at their own pace based on their individual effort. Workout profiles and courses are generally, but not always, presented with two-dimensional or three-dimensional graphical views and may include gamified environments or video. Virtual gamified environments often allow users to share the experience with, or race against, computer-generated users or other users who may also be connected online at the same time. Background sounds (e.g., nature or traffic) or audio (e.g., inconsequential music) are often played but do not directly correspond with the workout profile.

Beat-led workouts allow users to select independent audio sources (e.g., music or playlists) for a workout whereby a direct relationship exists between the beats per minute of an audio source and the desired pace or speed of the workout. Users often choose a preferred style or genre of music to specifically match a desired exercise outcome (e.g., beats per minute matches desired cadence or running pace) or modality (e.g., working out so that a cadence can be entrained). The rationale for a specific style or genre is usually based on personal preference, and the matching of pace is not related to or adjusted in response to topographical terrain.

U.S. Pat. No. 6,605,020 to Huang relates to a speed control device of an electric treadmill, the driving motor of which changes its speed according to the rhythm and the intensity of music while the operator adjusts his speed to the music rhythm so that the speed of music, motor and operator are identical to one another.

U.S. Pat. No. 8,690,735 to Watterson et al. relates to exercise devices that incorporate modules for receiving and decoding control signals embedded in multimedia programming for controlling various operating parameters of the exercise device in synchronization with the multimedia programming.

U.S. Patent Application Publication No. 2016/0059709 by Watterson relates to broadcasting media content to a display device, associating a downloadable fitness program with the media content with synchronization indicators to synchronize the downloadable fitness program with the media content, and controlling an operational parameter of an exercise machine based on physical characteristics of an environment depicted in the media content.

This background is not intended, nor should be construed, to constitute prior art against the present invention.

### SUMMARY OF INVENTION

The emotional topographies of entertainment sources are extracted by manual coding, automatic coding, real-time coding, biometric analysis, artificial intelligence (AI), user feedback or any combination of these. Emotion-led workout profiles are generated that are correlated with the emotional topography of each entertainment source. An emotion-led workout profile is then run in synchronization with the playing of its corresponding entertainment source to provide a user with an augmented workout.

Disclosed is a method for operating an exercise machine comprising playing an entertainment source while simultaneously controlling the exercise machine according to a workout profile that is synchronized with non-quantitative elements of the entertainment source.

Disclosed is an exercise machine configured to receive an entertainment source, receive a workout profile that is synchronized with elements of the entertainment source that are non-quantitative and non-literal, and play the entertainment source while controlling its settings according to the workout profile.

Disclosed is an exercise machine configured to receive a workout profile that is synchronized with elements of an entertainment source that are non-quantitative and non-literal, and control its settings according to the workout profile.

Disclosed is a non-transitory computer-readable medium storing computer-readable instructions which, when executed by a processor, cause an exercise machine to receive an entertainment source, receive a workout profile that is synchronized with elements of the entertainment source that are non-quantitative and non-literal, and play the entertainment source while controlling its settings according to the workout profile.

Disclosed is a method for generating a workout profile that is synchronized with an entertainment source, the method comprising identifying moments in the entertainment source that are each written to portray a different emotion or level of emotion, and tagging each corresponding moment in the workout profile with a different setting for an exercise machine, each setting relating to the corresponding emotion or level of emotion.

Disclosed is a system for generating a workout profile that is synchronized with an entertainment source, the system comprising a processor and a computer readable medium storing computer-readable instructions which, when executed by the processor, cause the processor to identify moments in the entertainment source that are each written to portray a different emotion or level of emotion, and tag each corresponding moment in the workout profile with a different setting for an exercise machine, each setting relating to the corresponding emotion or level of emotion.

Disclosed is a non-transitory computer-readable medium storing computer-readable instructions which, when executed by a processor, cause the processor to generate a workout profile that is synchronized with an entertainment source by identifying moments in the entertainment source that are each written to portray a different emotion or level of emotion, and tagging each corresponding moment in the workout profile with a different setting for an exercise machine, each setting relating to the corresponding emotion or level of emotion.

This summary provides a simplified, non-exhaustive introduction to some aspects of the invention, without delineating the scope of the invention.

### BRIEF DESCRIPTION OF DRAWINGS

The following drawings illustrate embodiments of the invention and should not be construed as restricting the scope of the invention in any way.

FIG. 1 is a flowchart of a process according to an embodiment of the present invention.

FIG. 2 is a schematic representation of a workout profile, according to an embodiment of the present invention.

FIG. 3 is a graphical representation of a segment of a work profile, according to an embodiment of the present invention.

FIG. 4 is a flowchart showing interactions between modules of the system, according to an embodiment of the present invention.

FIG. 5 is a schematic diagram of the system highlighting aspects of the motivation engine, according to an embodiment of the present invention.

FIG. 6 is a block diagram of a system, according to an embodiment of the present invention.

FIG. 7 is a block diagram of a system, according to an embodiment of the present invention.

### DESCRIPTION

### A. Glossary

Action potential—the ability of an entertainment source to stimulate exercise using non-quantitative aspects of the source. Other, quantitative aspects of the source may also contribute to the action potential.

Effort—this is the instantaneous, absolute amount of work per unit time (power) expended by a user during a workout. It is a physical quantity that is measured in Watts, for example.

Emotional topography—the changing emotions and attention-focusing aspects that are induced by an entertainment source when it is watched or listened to.

Emotion-led workout profile—a workout profile which corresponds at least in part to the emotional topography of an entertainment source to which it is synchronized. Where context is clear, it may simply be referred to as a workout profile or profile.

Entertainment source, or source—a digital media file used for entertainment, such as a movie, an episode of a series, a video clip, a soundtrack, a song, a podcast, a news report, a documentary, a series of still images, a series of video clips. The source may be streamed or provided in advance. The entertainment sources to which this invention relates are those created primarily for enjoyment and information purposes, and not for directly instructing a workout. As such, they may be considered to be non-instructional entertainment sources, non-workout entertainment sources, or non-workout-instructional entertainment sources.

Exercise instruction device—an electronic device that is configured to provide workout instructions that are synchronized with the emotional topography of an entertainment source. An exercise instruction device is usually not a mechanical device and does not physically interact with the user to provide the workout resistance to the user. An exercise instruction device may be an instructional exercise screen, an instructional exercise mirror, a smart TV, a tablet, a personal mobile communication device etc.

Exercise machine—a machine or apparatus that physically interacts with the user to provide the user with a workout.

Intensity—this refers to the user's instantaneous effort in comparison to the user's maximum effort. Intensity may be measured as a percentage, for example.

Micro-tag—a marker in a segment or a moment that corresponds to an instantaneous event in the entertainment source.

Module—can refer to any component in this invention and to any or all of the features of the invention without limitation. A module may be a software, firmware or hardware module, and may be located in a user device, an exercise machine or a server.

Moment—each segment of an emotion-led workout profile is divided into a series of moments. A moment may be a few seconds or more than a minute, for example. A moment may be, for example, a notable, relatively short duration in time compared to its segment, where workout potential or emotion is particularly elevated relative to the rest of the segment. For example, the climax of an exciting car chase or action scene from a film may be identified as a moment. A moment may define, for example, a constant, but relatively high level of resistance. Other moments may be low intensity moments, flat moments or transition moments. Moments may be micro-tagged with texture elements.

Processor—refers to any electronic circuit or group of circuits that perform calculations, and may include, for example, single or multicore processors, multiple processors, an ASIC (Application Specific Integrated Circuit), and dedicated circuits implemented, for example, on a reconfigurable device such as an FPGA (Field Programmable Gate Array). The processor performs the steps in the flowcharts, whether they are explicitly described as being executed by the processor or whether the execution thereby is implicit due to the steps being described as performed by code or a module. If the processor is comprised of multiple processors, they may be located together or geographically separate from each other. The term includes virtual processors and machine instances as in cloud computing or local virtualization, which are ultimately grounded in physical processors.

Real-time—this means that as one action is occurring, another action is occurring in response to it and at the same time, subject to inherent time lags due to electronic and mechanical limitations. The actions may appear to a human to be simultaneous, or to be close enough together that their occurrences are, for substantially all intents and purposes, as good as simultaneous.

Segment—a portion of an emotion-led workout profile. Each emotion-led workout profile is divided into a series of segments. A segment is, for example, a notable window of time where workout potential or emotion is particularly elevated for an extended period. For example, an exciting car chase or action scene from a film can be identified as a distinct reference segment. Other segments may be less intense or less dynamic, and there may be flat segments or transition segments. A segment duration may be shorter than 1 minute or more than 10 minutes, for example. Segments are divided into moments that are tagged with exercise-based settings such as resistance settings, cadence settings, slope settings or changes in these.

System—refers to a system for creating emotion-led workout profiles and running or directing the corresponding workouts in synchronization with an entertainment source.

Tag—a marker in a moment that represents a setting or a change in setting of an exercise machine. The setting may be, for example, the resistance, cadence or slope.

### B. Rationale

The inventors have recognized a behaviour that often accompanies traditional instructor, course and beat-led workouts, whereby users add a second screen or entertainment source, independent of the workout content, for entertainment and to motivate and/or distract themselves. This additive element most often takes form as streaming content, movies, TV shows and series, video clips, music, audio books and podcasts. In a multiple screen/source scenario, one screen/source directly relates to the traditional workout and the other allows the user to play entertainment content of their choice.

The inventors have identified that it is the emotional arousal and attention focusing used in entertainment media that are desired and useful to exercisers for the purpose of motivation and/or distraction. The entertainment industry, in particular through film, is expert in capturing attention as well as stimulating and leading an audience through emotional highs and lows depicted by a story, scene by scene. The relationships between stimulation, captured attention and response, however, are not literal, proportional or linear.

For example, a slow paced, intense melody or beat that might accompany multiple angled and interplayed shots of a character in motion can in fact relate to high suspense and drama (e.g., the classic two note leitmotif of the Jaws movies and a signal of impending danger). High suspense and drama result in a highly stimulating effect for the audience and present a major source of ‘action potential’ and, in turn, ‘motivation’ for a workout. In the observed behaviour this manifests as moments of increased cadence and effort from the user in direct response to the dynamics of the scene. This is counter to the application of a traditional beat-led workout where faster beats alone are equated to greater cadence but not necessarily effort. It also eliminates the need for explicit instruction and terrain from instructor-led and course-led solutions. In fact, the key roles of the artist, editor and director teams are to evoke a varying emotional response by bringing the story to life and the user follows intuitively.

A contrasting example would be the John Wick movies where intense and prolonged action with scenes depicting violent fighting can lead a rider to become ‘stunned’ whereby cadence is greatly reduced, although high power can be maintained. Alternatively there are commonly ‘transition’ scenes within a movie where an upbeat soundtrack accompanies simple motion, as in Saturday Night Fever where the main character is walking along the high street. These scenes offer high cadence potential but limited ability to increase power/workload. Scenes such as horizon views or ‘nature’ scenes are most suited to low cadence, low power efforts.

Entertainment media exhibit intentionally designed and combined non-quantitative features and artifacts that the inventors have specifically identified, analyzed and structured into a rule-based logic. For example, signature audio characteristics such as a leitmotif are created within the accompanying movie score and combined with visual scene cutting techniques such as the Kuleshov effect (character eye-line, action panning, point of view, etc.). The specific combination in any moment offers a particular intensity and effort to be demanded from the user. There are many tactics that artists and filmmakers deploy to deliver a desired emotional state of arousal. Users will often seek out certain show or movie genres to support a workout (e.g., action, thriller, or suspense) or seek out specific sequences (e.g., as car chases, fight scenes or stunts) that have been carefully formulated to maximize the stimulation of the audience. These scenarios have been attributed with specific codes by the inventors to create a wide variety of exercise experiences. Furthermore, nature scenes and focused dialogue/narrative have been proven to neutralize an emotional state to promote a feeling of calm despite the presence of other external stimuli.

A traditional beat-led workout would not pick up on the presence of the leitmotif, especially if it occurs at the same musical tempo and volume level as an immediately preceding soundtrack. Traditional quantitative analyses of entertainment sources would not pick up on the suspense invoked by the absolute silence of a protagonist tentatively advancing into a dangerous, dimly-lit room. Likewise, particular words spoken in a dialog without changing the intensity of the conversation may trigger an emotional response that would not be captured by quantitative analysis.

Numerous studies have demonstrated the psychophysiological response to emotion-eliciting, emotion-neutralizing and attention-acquiring stories, scenes, and sequences. The various levels of arousal response can be demonstrated by changes in biological signals (e.g., heart rate, heart rate variability, blood pressure, breathing rate, breathing gas composition, galvanic skin response). The inventors have discovered that the changes observed are not dissimilar to what would normally be associated with varying activity during exercise. The present disclosure capitalizes on this energetic action potential. An emotional response therefore presents an opportunity to align resistance from connected exercise equipment, or from any workout regime, with the energizing, focusing, or calming emotional stimuli of any entertainment source.

To date, there has been no solution offered to extract and exploit the complex relationship between emotional stimulation and the potential to directly influence effort during a workout through controlled resistance of exercise equipment.

### C. Exemplary Embodiments

The inventors have developed a module, called the ‘motivation engine’, and associated method to produce exercise workout profiles based on content from external entertainment sources (e.g., film, TV show, video clip, streaming service). The resultant workout profile represents an emotional topography of the entertainment source. Each unique workout profile has, for example, a series of segments, each segment having a series of moments, and each moment having exercise machine settings and assigned micro-tags. The moments represent exercise and interactive elements correlated with the entertainment source, e.g. resistance, cadence, slope and sensory texture. The motivation engine creates a unique digital workout file, timestamped by segment, moment and micro-tags, associated with the analyzed entertainment content source. The entertainment source and the emotion-led workout profile are then output or ‘played’ in synchronization.

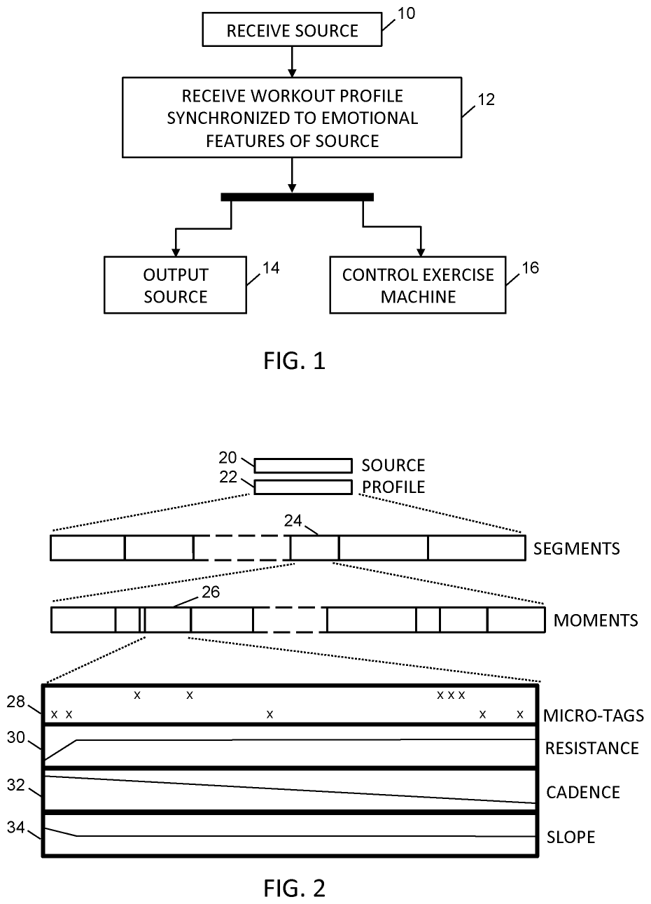

Referring to the exemplary embodiment of FIG. 1, which relates to the control of an exercise machine, an entertainment source is received, in step 10, by a communications device such as a smartphone or tablet, or other electronic device with similar capabilities. A processor controlling the communications device receives the entertainment source from a streaming service or as a complete file in advance of the workout that is to be performed by the user. The emotion-led workout profile is received by the communications device under control of the processor in step 12. Again, the processor may receive the emotion-led workout profile from a streaming service or it may receive it in advance of the workout that is to be performed.

Steps 14 and 16 occur in synchronization. In step 14, the entertainment source is output, or played, by the processor. The output device used to output the source may be the communications device used to receive the source and the workout profile. In other embodiments, the output device may be a screen that is part of the exercise machine. In step 16, the emotion-led workout profile is used to control the exercise machine. This may be achieved by the processor in the communications device loading or feeding the workout profile into the exercise machine and starting it in synchronization with starting the output of the entertainment source. Alternately, the starting of the workout profile on the exercise machine may trigger the start of the synchronized playback of the entertainment source.

Referring to FIG. 2, the details of an exemplary workout profile 22 are shown. The profile 22 has the same duration as the entertainment source 20 to which it corresponds. The entertainment source 20 may be, for example, a complete episode of a series or a partial episode.

Each workout profile is divided into multiple segments 24 end-to-end, and each segment is divided into multiple moments 26 end-to-end. A moment 26 is defined by micro-tags 28 and exercise machine settings 30, 32, 34. The exercise machine settings may be in the form of a graph or a numerical table of values against time. The parameters that may be controlled by the settings include, for example, resistance 30, cadence 32 and slope 34. Other parameters may be controlled by other settings that are included in the moment. A profile may be configured to be specific to a particular exercise machine or it may be universal, in that it has settings for multiple types of exercise machine and a particular exercise machine only uses the settings that are relevant.

Each moment 26 may have a single, specified value for the resistance setting for that moment. During an initial time period of the moment, the resistance setting will gradually change from what it was to the single value. The initial time period may be of a fixed duration, or it may be determined by the rate at which the resistance setting can comfortably and practically be changed. The same applies to the other parameters of the exercise machine that can be set. In other cases, the resistance or other parameters may be changed continually throughout the moment, as in this example of the cadence 32.

The micro-tags 28 correspond to instances of the entertainment source where there are momentary events, such as a gunshot, an explosion, a car crash, a surprise, a shock, a sound effect, a sudden realization, etc. These micro-tags 28 are used to control a sensory texture layer of the exercise machine. For example, the electric motor or level of resistance may be made to deliberately stutter to cause a vibration that is felt by the user. The application of sensory micro-tagging creates a texture layer that can be experienced as momentary interruptions of the exercise stimulus to increase the immersive value of the whole exercise experience where the connected exercise machine permits. In some cases, a micro-tag may coincide with the start of a moment that demands a high intensity and high cadence.

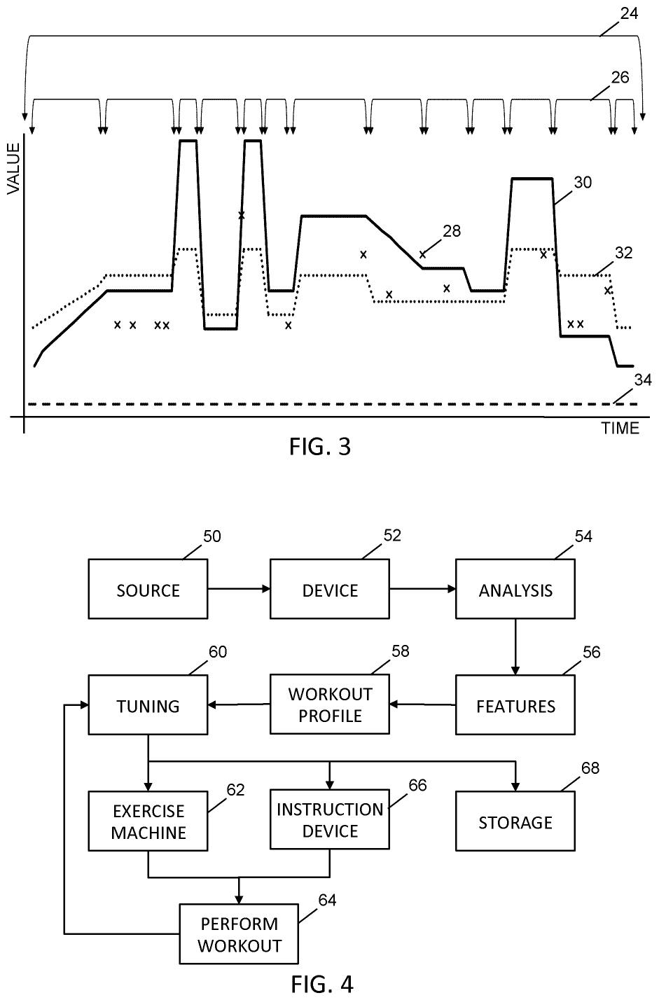

FIG. 3 shows a graphical example of a segment 24, which is divided into multiple moments 26 end-to-end. The time axis is along the bottom and the value axis is along the left of the figure. The value axis is in arbitrary units, which may be different for each parameter plotted. The resistance 30 and cadence 32 vary considerably throughout the segment 24, but, for the most part, have a steady value or steady gradient throughout each moment 26. The slope setting 34 is shown to have a relatively low, static value for the duration of the segment 24. The values and gradients of the exercise-related parameters of the exercise machine may be described as tags. Exemplary tags may be 110% of functional threshold power (FTP) and a specific call for high cadence such as 100 revolutions per minute.

The micro-tags 28 are also shown to have different values, which may correspond to the loudness or emotional impact of the corresponding feature in the entertainment source. In some embodiments, there may be a time aspect to each micro-tag, which governs how long the sensory texture activation of the exercise machine lasts. For example, the vibration may last, say, a tenth of a second, or it may exceed 2-3 seconds, for example where multiple concurrent explosions occur. The micro-tags relate to immersive elements of the workout profile, and while they are useful for that purpose and for helping to define the emotional topography of the entertainment source, they are individually too short to base an exercise-related parameter of the exercise machine on. The sensory layer is therefore a non-exercise sensory layer. Depending on the exercise machine, the sensory texture layer may not be available, or it may be available and possible to be turned on and off by the user.

While the segments may be tagged in many ways, there are four main categories of tagging: high power and high cadence; high power and low cadence; low power and high cadence; low power and low cadence. Each of these categories may have its own matrix of finer settings. A flat segment would be akin to riding on a flat, featureless road and may be a consistent application of one or both of the latter two tagging categories. Sometimes it is of value to a user to ride a flat segment and there is benefit in doing this with a movie as distraction versus without, even if there are little to no changes in the effort required.

Referring to FIG. 4, the main modules or components of an exemplary system are shown. To start, an entertainment source 50 is identified. The source may be an audio or video source, such as a song, a movie, a TV show, a podcast, etc. and is in the format of a digital file. The source is then downloaded or otherwise transferred to an electronic device 52, such as a computer, smartphone, smart device, tablet, smart TV, laptop, etc. The source is then fed into an analysis module 54, which may be in the device 52 or in another electronic device such as a server. The analysis module 54 analyzes the raw signals in the digital file for the entertainment source, with or without human intervention.

The features module 56 represents the features that are extracted from the digital file. At least some of the features are non-quantitative, such as genre, excitement, conflict, anger, surprise, happiness, suspense, attraction, calmness and other emotion-triggering aspects of the entertainment source such as a chase, a car chase, a fight, an argument, an escape, a capture, a battle, a victory, a goal, a cheer, a celebration, a joke, an insult, a conversation, a facial expression, a look between two characters, a realization, a shock, an accident, a breakthrough, silence, stillness, speed, nature, a birth, a marriage, a promotion, a leitmotif or a symbol. The non-quantitative elements represent an emotional topography of the entertainment source and may include one or more scenes written to portray suspense, happiness, anger, peace, fear, danger, skill, success, passion, justice, adventure, excitement, surprise, disbelief, astonishment or hatred. These features are also non-literal, in that they do not explicitly instruct the consumer or user to perform a particular action, and they may be referred to as non-instructional features. Some of the features may be quantitative, such as musical tempo for a song or soundtrack, or volume.

The emotion-led workout profile 58 is generated using the features that are present in the features module 56. The profile is generated by defining settings for parameters of an exercise machine, for example the slope gradient of a running machine or the resistive force of an elliptical machine. At least some of the settings and changes in settings are synchronized with the emotional topography of the entertainment source. That is, some of the settings of the exercise-related parameters in the emotion-led workout profile are correlated with non-quantitative features of the entertainment source.

The tuning module 60 allows a user, other person or AI machine to adjust the emotion-led workout profile. For example, the settings in the workout profile may be smoothed, for example if changes are too frequent or too abrupt. Settings may be tuned according to a user's preference. For example, the whole or part of the workout profile may be scaled in intensity according to the current ability or desire of the user. The sensory texture layer may be toggled on or off depending on the user's preference. Settings may be tuned to a particular cohort of users.

The workout profile, which may be tuned or not, is then uploaded or otherwise transferred to an exercise machine 62. The exercise machine may be, for example, a rowing machine, a treadmill, an elliptical machine, a smart bike, etc.

Block 64 represents the performance of the workout by the user on the exercise machine. The workout may be rowing, running or cycling, for example. The workout profile, which has been input into the exercise machine, is started at the same time as the entertainment source is started. The exercise machine therefore runs the workout profile in synchronization with the entertainment source. While the workout profile is controlling the parameters of the exercise machine, the user may optionally tweak the settings of the exercise machine. These adjusted settings may be fed back to the tuning module 60 in order to refine the workout profile for a subsequent workout by the same user, or for provision to another user. Feedback from cohorts of users may be averaged to provide an improved workout profile for other members of the cohort. In some embodiments, the adjusted settings may be transferred directly from the exercise machine to the tuning module 60. The adjusted settings may be downloaded from the exercise machine to an application on the user's electronic device (e.g. smartphone) and fed back by the user via the application to the tuning module 60. In other cases, the user may adjust the workout profile by manually making inputs to the tuning module 60.

In an alternate path, the workout profile, which may be tuned or not, may be downloaded or otherwise transferred to an exercise instruction device 66. The exercise instruction device may be a user's smart device, smartphone, laptop, smart TV, desktop, fitness instruction screen, graphical interface etc. The workout profile includes voice commands, textual commands, numerical commands or visual commands for performing the workout. These commands are defined in the workout profile so that they correspond to the emotional topography of the entertainment source. In this scenario, block 64 represents the performance of the workout by the user without an exercise machine, or on an exercise machine that does not accept the input of an emotion-led workout profile. The workout may be rowing, running, cycling, step or aerobics for example. The workout profile, which has been input into the exercise instruction device, is started at the same time as the entertainment source is started. The exercise instruction device therefore runs the workout profile in synchronization with the entertainment source.

Storage 68 is a non-volatile memory module, for storing emotion-led workout profiles and identifiers of the entertainment sources to which they correspond. The entertainment sources may also be saved in the storage. In some embodiments, different emotion-led workout profiles may be stored for the same entertainment source. For example, different workout profiles may be stored for different cohorts of users. Also, different workout profiles may be stored for the different sets of fans of the teams in a sports match. Storage 68 may store the tuned or tailored workout profiles for future use, either by the same or different users. The storage module 68 may be set up so that it serves as a library or marketplace for profiles that may be accessed, rented or purchased by the users. The storage module 68 may be in a remote server accessible via the internet, in a user's mobile electronic communication device or other personal electronic device, in an exercise device or in an exercise machine. The storage module may be replicated in multiple locations.

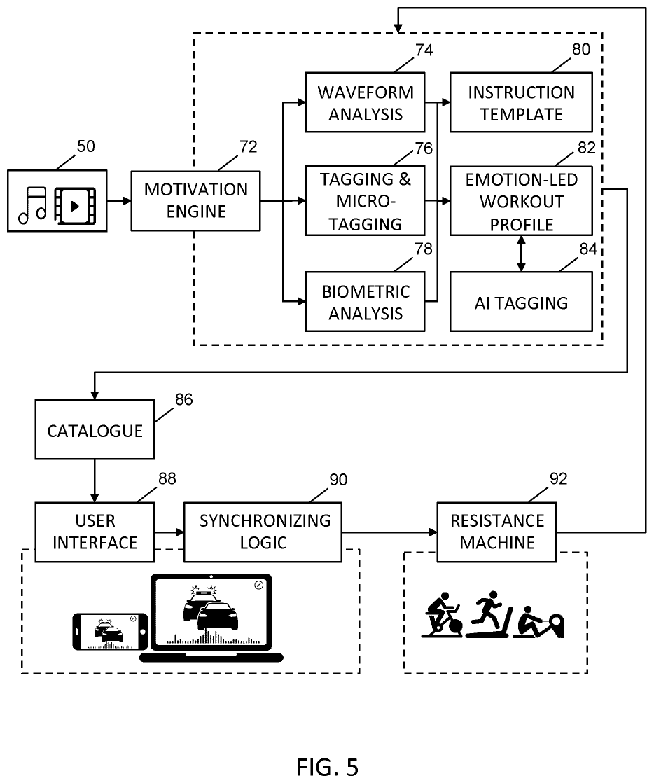

FIG. 5 shows exemplary methods for creating emotion-led workout profiles for entertainment sources 50, such as songs, movies, TV shows, video clips, podcasts, audio books etc. After an entertainment source 50 has been selected, it is passed to the motivation engine 72, which may include components similar to the analysis module 54, the features module 56, the workout profile module 58 and the tuning module 60 of FIG. 4.

The analysis using the motivation engine 72 includes audio waveform analysis 74. This may identify, for example, instances where there are gunshots, a period of silence, or where there is a leitmotif. Tagging and micro-tagging 76 includes genre classification, scene tagging, section tagging, segment tagging and micro-tagging of the moments. The tagging relates to different emotional aspects of the entertainment source. Manual tagging may be achieved using a bespoke profile generator software tool, which allows a person to input the tags in the workout profile at specific points that correspond to events in the entertainment source. Automatic tagging may also be used, where a machine learning or AI algorithm 84 is trained to ingest the source with or without human assistance. Real-time tagging may be used, where the software ingests live content such as sporting events to deliver an immediate resistance setting that matches the emotional topography of the changing events. The events may be a goal, a foul, an extraordinary play, a change in excitement level of the commentator, a crowd's reaction, a crowd's chant or a Mexican wave, for example. There may be different workout profiles for each team's supporters. When live streaming a live event, or when simultaneously streaming a workout and its corresponding entertainment source to a plurality of users, the changes some of the users make to their exercise machines may be captured and fed back to a server running the motivation engine, where they are incorporated in real-time into the workout profile for the other users to experience in real time. Micro-tagging includes the identification of pinnacle events, such as gunfire, an explosion, a crash, a sudden noise, a punch, a kick, a stab, a pothole that is driven over or a fall.

Further to the extraction from the source of creative audio-visual features, the capture in module 78 of biometric signals associated with the arousal response from an individual/audience have validated that emotion-led workout profiles can be further enriched. This capture can be achieved by one or more biometric sensors during a ‘watch only’ setting i.e., in the absence of exercise routines, or as users naturally respond to the different emotional stimuli of a source that is presented during a standardized exercise routine. These biometric signals or measurements can be captured separately and used to further define the emotion-led workout profile, to further enhance the individual experience of real-time resistance. This may be done manually, automatically (proportionally or linearly) or using the AI algorithm 84.

Each of the above methods can be applied together or independently to define the workout profile and texture layers.

While the emotional topography of an entertainment source is non-quantitative, some of the effects of the entertainment source on users are measurable. For example, a biometric response is quantifiable, and the work expended by a user when performing an emotion-led workout profile is measurable. The emotion-led workout profiles created by the system are quantitative, and their effects on the workout performance of users can be measured if desired.

One, some or all of the above techniques are used for generating the instructional template 80 for a workout instruction device and for generating the emotion-led workout profile 82. Both the instruction template and the emotion-led workout profile may be referred to as motivation files. The instruction template may also be referred to as an emotion-led workout profile.

All workout profiles are stored in the form of a curated catalogue 86 in the storage module 68 (FIG. 4). The profiles are synchronized with the entertainment source using synchronization logic 90 whenever the user so chooses, via a software application that provides a user interface 88. This allows the user to exercise on a resistance machine 92 while consuming the entertainment source and simultaneously performing an emotion-led workout.

After they have been created, each workout profile may be analyzed further to summarize various aspects of it and its related entertainment source. For example, the entertainment source may be assigned a workout potential or density, which may be expressed as a percentage that represents the total workout time relative to total duration of the entertainment source. For ratings that are below 100%, then it is possible for the portions that are not used for generating the workout profile to be removed from the entertainment source.

Another classification parameter of the workout profiles may be the number of distinct segments, where each segment corresponds to a standalone workout. Alternately, a workout may be defined as a group of segments, and the classification may relate to the number of workouts in the workout profile, or for the corresponding entertainment source.

While all segments have workout value, some are more valuable than others from an exercise science point of view. For example, segments that have a wider range or variability between the lowest intensity and the peak intensity are considered to be more valuable. The bigger the intensity range within a segment, the greater the potential to efficiently increase user fitness. Each workout profile, or each segment within a profile, may therefore be given a rating that is calculated from the ratio of peak intensity to the valley intensity, or the difference between them, and the number of times that the peaks and valleys are present.

Another rating that may be given to a workout profile or its corresponding entertainment source is the exercise modality, or type. This represents a comparison of the emotion-led workout profile to a standard workout modality, e.g., recovery, Tempo, interval, etc. The count of the sensory texture events in the segments or the workout profile may also be expressed, to give an indication of how immersive the workout profile is.

The application of the motivation engine enables a certain intelligence as to the workout value of any entertainment source. Specific metrics may be associated with perceived effort to actual effort ratios, workout quality based on applied exercise science, and workout density based upon how many distinct workout segments can be identified from any single entertainment source. An advantage of some embodiments of the invention is that by reducing the perceived effort to actual effort ratio, then users can become fitter than they would if they used non-emotion-led workout profiles, or they could become as fit more efficiently, i.e. using shorter workout durations.

The perceived amount of work expended during a workout may be determined qualitatively by the user. For example, the user may rate the workout as more difficult than expected, about the same as expected, or easier than expected. In other embodiments, the user may input a numerical value of the perceived amount of work. Other techniques may be used for capturing the user's perceived amount of work. For example, the well-established standard known as the Borg Scale for rating of perceived exertion may be used. Additionally there is another known method called the Net Promoter Score that may be used to obtain an overall reaction to the completed workout each time. The user may also be provided with simplified versions of these by the system.

Furthermore, sections or clips can be extracted from various entertainment sources to construct classic workout structures such as intervals (short, repeated efforts), ramps or physiological tests such as the FTP test. Such a compilation of entertainment sources is also referred to as an entertainment source.

All these aspects may be used to describe a workout profile and its corresponding entertainment source in the catalogue 86. This then allows users to filter the available profiles and entertainment sources according to their preferences. Any segment that offers specific exercise value e.g., interval, Tempo, recovery, etc. is highlighted for the benefit of certain users who might require that detail. There is no limit envisaged on the type or quantity of entertainment sources that can be used to create emotion-led workout profiles. The ongoing interaction with the catalogue by the user will produce insight into what entertainment sources are preferred or receive greatest levels of adherence and hence can drive recommendations for other entertainment sources to be run through the motivation engine 72.

To support gamification of the system, users' best times or achievements may be fed into the catalogue 86, which may be configured to display a leadership table for each segment or each workout profile. In some embodiments, users are permitted to compile their own entertainment sources from existing sources, or to submit their own to the catalogue. They may also be able to submit their own workout profiles based on recordings of the parameters of their own exercise machines while exercising when watching or listening to an entertainment source.

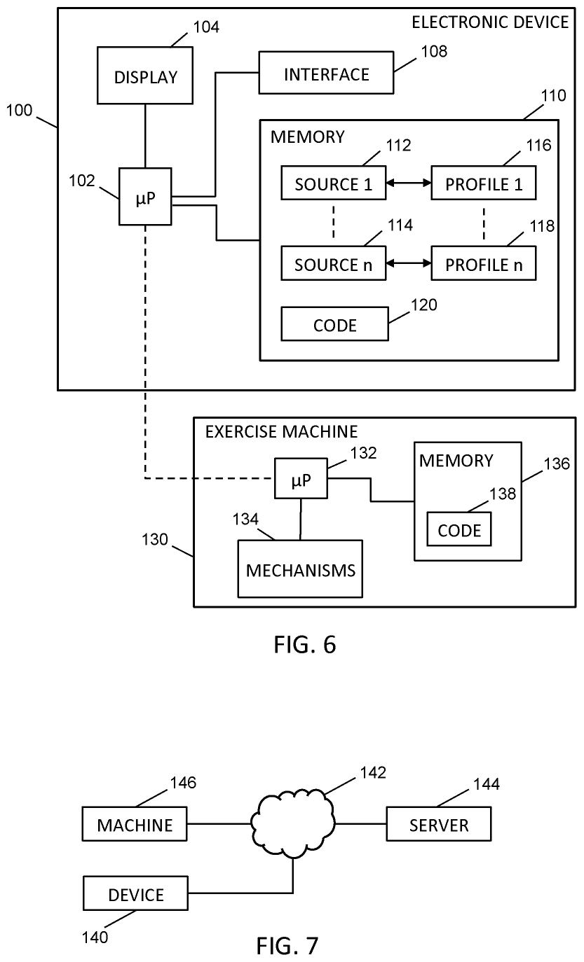

Referring to FIG. 6, an exemplary system is shown, which includes an electronic device 100 connectable to an exercise machine 130. The device 100 has a processor 102 connected to a display 104, user interface 108 and memory 110. The interface 108 and display 104 may be one and the same component, such as a touchscreen. The memory 110 may be divided into one or more units, of one or more different types. The memory 100 stores computer-readable identifications 112, 114 of entertainment sources 1-n, or it stores the complete digital files for the entertainment sources. The memory also stores the emotion-led workout profiles 116, 118 that correspond to the entertainment sources 112, 114, and a record of which source corresponds to which profile. Also stored in the memory 110 is computer readable code 120, which, when executed by the processor 102, causes the device 100 to perform one or more functions of the system.

The exercise machine 130 has a processor 132 that controls the mechanisms 134 of the exercise machine to change its resistance, slope or cadence, or to output a sensory texture event. The memory 136 is connected to the processor 132 and stores computer-readable instructions in the form of code 138, which, when executed by the processor 132, cause the exercise machine 130 to perform one or more functions of the system.

The code 120 may be in the form of an independent software application with a graphical interface on the display 104 of a device 100 such as smartphone, tablet or smart TV with access to a digital library (112, 114, 116, 118). The digital library may be local, i.e. in the device 100, or remote, i.e. in another device connected wirelessly or with a wired connection to the device 100.

In another embodiment, the components of FIG. 6 may all be combined in a single exercise machine.

Alternately, the code 120 may be embedded in an existing software interface such as a streaming subscription service. The existing software application communicates directly with the exercise machine 120 to provide resistance settings defined by the emotion-led workout profile 116, 118.

FIG. 7 shows an arrangement of the system in which the user's electronic device 140 is connected via the internet 142 or other network to a server 144 that provides a streaming service. The server 144 streams the selected entertainment source to the user's device 140 while at the same time streaming the emotion-led workout profile via the internet 142 to the exercise machine 146. It is also possible that the emotion-led workout profile is downloaded in its entirety to the exercise machine 146 or the device 140 before the streaming of the entertainment source starts. The main requirement is that the running of the exercise machine 146 according to the emotion-led workout profile is synchronized with the streaming of the entertainment source.

Should a user not have access to connected training equipment such as an exercise machine, then the emotion-led workout profile can also be represented by textual, numerical, graphical, audio, visual or audiovisual instructions. These instructions may be received via a screen or by speakers or headphones. The instructions can also be added as part of the user interface 108 as an information layer that is coordinated with the associated entertainment source to support in-the-moment feedback and guidance. Guidance may be, for example, for the user to set a non-connected exercise machine to a particular setting, which may be a resistance setting, a slope setting or a cadence setting.

The system described herein permits users to efficiently use only one screen and receive exercise value from their entertainment source of choice beyond that which would be directly proportional, linear or literal in relation to the source. Users' choices may be specifically distracting or relative to their own exercise potential. This emotion-led workout method broadens the utility of all entertainment media for users seeking new options and to enable users to receive and record benefit from emotion-led stimuli. A benefit to the user is that the action potential of an entertainment source can induce greater levels of effort for a relatively lower perception of said effort. This is a fundamentally valuable attribute to all individuals seeking to maximize the efficiency of their workout time. The catalogue of entertainment sources for which there are emotion-led workout profiles may include recommendations of exercise value for each of the sources. For example, the action potential of a source may be classified as high, medium or light, or it may be defined as a numerical value. A classification or numerical value may be given for the averaged actual effort as a ratio to average perceived effort for each source.

While this invention is directed towards exercise enthusiasts of all levels where the two-screen behaviour has been observed, it opens up accessibility to workouts for many more user groups including children or sedentary adults who might not be attracted to traditional, literal workouts (instructor, course or beat led). This represents a new market opportunity for workout administrators and for the manufacturers of increasingly connected training equipment. Additionally, this supports the expansion of the entertainment industry with new ancillary revenue through granting of exercise rights to certain content as well as supporting streaming platforms looking to increase consumer engagement with content libraries or to reduce churn of membership. The emotion-led workout profiles may be sold as exercise rights, either separately or bundled with the rights to the corresponding entertainment sources. While the entertainment sources used for the system described herein are non-instructional entertainment sources, it is envisaged that future entertainment sources may be written in such a way that they can deliberately but indirectly lead a workout via incorporation of an intentionally designed emotional topography that is particularly suitable for an emotion-led workout.

### D. Variations

The workout profile may have a different duration to the entertainment source. For example, the workout profile may start before or after the entertainment source, and finish before or after the entertainment source finishes. Differences in duration of the workout profile and entertainment source may be used for warm-up or cool-down periods, for example.

Referring back to FIG. 1, the processor may be in an exercise instruction device, which may be a communications device such as a smartphone or tablet, or it may be a television. In these cases, the exercise instruction device is configured to instruct the user on how to adjust the settings of an exercise machine without itself being connected to the exercise machine. In some embodiments, the output of the workout profile may be on the same output device as that used to output the entertainment source. For example, the output of the profile may be textual or numerical, and displayed on the screen that is displaying the entertainment source. In another example, the output of the profile may be verbal instructions that are played over the audio track of the entertainment source. These instructions are then followed by the user to adjust the settings of the exercise machine on which the workout is being performed.

Different people react to emotional triggers differently, and so in some embodiments there may be different emotion-led workout profiles for the same entertainment source, each profile corresponding to a different emotional type of person.

The sensory texture layer may in some embodiments include the activation of lights on the exercise machine, the change in intensity of lighting on the exercise machine, a flashing of lighting on the exercise machine, or the change in colour of lighting on the exercise machine.

Throughout the description, specific details have been set forth in order to provide a more thorough understanding of the invention. However, the invention may be practiced without these particulars. In other instances, well known elements have not been shown or described in detail and repetitions of steps and features have been omitted to avoid unnecessarily obscuring the invention. Accordingly, the specification is to be regarded in an illustrative, rather than a restrictive, sense. In general, unless otherwise indicated, singular elements may be in the plural and vice versa with no loss of generality. The detailed description has been presented partly in terms of methods or processes, symbolic representations of operations, functionalities and features of the invention. Such processes may be controlled by programming instructions stored in one or more tangible or non-transient media readable by a computer or processor. The code modules may be stored in any computer storage system or device, such as hard disk drives, optical drives, solid state memories, etc. Computer readable instructions may be broken down into blocks of code or modules. The methods may alternatively be embodied partly or wholly in specialized computer hardware, such as ASIC or FPGA circuitry.

Embodiments, depending on their configuration, may exhibit all or fewer than all of the advantages described herein. Other advantages not mentioned may be present in one or more of the embodiments. Features from any of the embodiments may be combined with features from any of the other embodiments to form another embodiment.

It will be clear to one having skill in the art that further variations to the specific details disclosed herein can be made, resulting in other embodiments that are within the scope of the invention disclosed. Two or more steps in the flowcharts may be performed in a different order, other steps may be added, or one or more may be removed without altering the main function of the invention. Flowcharts from different figures may be combined in different ways. Modules may be divided into constituent modules or combined into larger modules. All parameters and configurations described herein are examples only and actual ones of such depend on the specific embodiment. Accordingly, the scope of the invention is to be construed in accordance with the substance defined by the following claims.

1. A method for operating an exercise machine comprising:
playing an entertainment source that has non-quantitative elements to which a biometric response has been determined; while
controlling the exercise machine according to a workout profile that is based on the biometric response and synchronized with the non-quantitative elements.

2. The method of claim 1, wherein the non-quantitative elements represent an emotional topography of the entertainment source.

3. The method of claim 1, wherein the non-quantitative elements include a scene that is written to portray suspense, happiness, anger, peace, fear, danger, skill, success, passion, justice, adventure, excitement, surprise, disbelief, astonishment or hatred.

4. The method of claim 1, wherein the non-quantitative elements include a chase, a car chase, a fight, an argument, an escape, a capture, a battle, a victory, a goal, a cheer, a celebration, a joke, an insult, a conversation, a facial expression, a look between two characters, a realization, a shock, an accident, a breakthrough, silence, stillness, speed, nature, a birth, a marriage, a promotion, a leitmotif, a symbol or a combination selected therefrom.

5. The method of claim 1, wherein the workout profile is further synchronized with a quantitative element of the entertainment source.

6. The method of claim 1, wherein the workout profile is further synchronized with instantaneous elements of the entertainment source, the method further comprising operating the exercise machine to provide sensory outputs that coincide with the instantaneous elements.

7. The method of claim 6, wherein the instantaneous elements include a gunshot, an explosion, a crash, a sudden noise, a punch, a kick, a stab, a pothole that is driven over, a fall or any combination selected therefrom.

8. The method of claim 1, wherein controlling the exercise machine comprises adjusting a resistance of the exercise machine, a cadence of the exercise machine or a slope of the exercise machine to a setting defined in the workout profile.

9. The method of claim 1, comprising receiving, by the exercise machine, the workout profile from a streaming service.

10. The method of claim 1, comprising receiving, by the exercise machine, the workout profile from a mobile electronic communications device.

11. The method of claim 1, comprising receiving, by the exercise machine, the workout profile in its entirety before said controlling the exercise machine starts.

12. The method of claim 1, comprising the exercise machine receiving the entertainment source and the workout profile, wherein said playing is carried out by the exercise machine.

13. The method of claim 1 comprising creating the workout profile by:
identifying the non-quantitative elements;
determining the biometric response; and
tagging each moment in the workout profile that corresponds to one of the non-quantitative elements with a setting for the exercise machine, each setting based on the biometric response.

14. The method of claim 1, comprising outputting textual, numerical, vocal, audio, visual, audiovisual or graphical commands for controlling the exercise machine according to the workout profile.

15. An exercise machine configured to:
receive a workout profile that is based on a biometric response that has been determined for elements of an entertainment source that are non-quantitative; and
control its settings according to the workout profile.

16. The exercise machine of claim 15 configured to:
receive the entertainment source; and
play the entertainment source while controlling its settings according to the workout profile.

17. The exercise machine of claim 15, further configured to generate sensory outputs corresponding to micro-tags in the workout profile that represent instantaneous elements of the entertainment source.

18. The exercise machine of claim 15, further configured to:
record adjustments made to the settings by a user when the user is exercising on the exercise machine; and
transmit said adjustments to a mobile electronic communications device or a server.

19. The exercise machine of claim 15, further configured to receive the entertainment source and the workout profile via a streaming service.

20. The exercise machine of claim 19, wherein the entertainment source is a live stream of a live event and the workout profile is generated in real-time.

21. A non-transitory computer-readable medium storing computer-readable instructions which, when executed by a processor, cause an exercise machine to
receive an entertainment source that has non-quantitative elements to which a biometric response has been determined;
receive a workout profile that is based on the biometric response; and
play the entertainment source while controlling its settings according to the workout profile and in synchronization with the non-quantitative elements.

22. The non-transitory computer-readable medium of claim 21 wherein the computer-readable instructions, when executed by the processor, further cause the exercise machine to generate sensory outputs corresponding to micro-tags in the workout profile that represent instantaneous elements of the entertainment source.

23. The non-transitory computer-readable medium of claim 21 wherein the computer-readable instructions, when executed by the processor, further cause the exercise machine to:
record adjustments made to the settings by a user when the user is exercising on the exercise machine; and
transmit said adjustments to a mobile electronic communications device or a server.

24. The non-transitory computer-readable medium of claim 21 wherein the computer-readable instructions, when executed by the processor, further cause the exercise machine to receive the entertainment source and the workout profile via a streaming service.

25. A method for generating a workout profile that is based on and synchronized with an entertainment source, the method comprising:
identifying non-quantitative elements in the entertainment source;
determining a biometric response to the entertainment source; and
tagging each moment in the workout profile that corresponds to one of the non-quantitative elements with a setting for an exercise machine, each setting based on the biometric response.

26. The method of claim 25 comprising:
identifying instantaneous elements in the entertainment source; and
micro-tagging the workout profile at instances corresponding to the instantaneous elements.

27. The method of claim 25 wherein the identifying, the determining and the tagging are performed by:
manual input to a software application;
the software application automatically or automatically by another software application;
measuring biometric responses of a consumer of the entertainment source and inputting said responses to the software application or a further software application;
an artificial intelligence application; or
any combination selected therefrom.

28. The method of claim 25 comprising tuning the workout profile to:
smooth it;
adjust it according to a user request; or
incorporate adjustments to exercise machine settings that are made by other users who have exercised to the workout profile.

29. The method of claim 25 comprising:
measuring a total amount of work expended by a user when the user performs a workout corresponding to the workout profile;
determining a perceived amount of work that is perceived by the user to be expended by the user during the workout; and
calculating a rating for the workout profile that represents the amount of work expended compared to the perceived amount of work.

30. The method of claim 29 comprising storing, in a catalogue:
the workout profile;
the entertainment source or an identification of the entertainment source; and
a classification of the workout profile that signifies an amalgamation of said ratings for a plurality of users.

31. The method of claim 25, wherein the entertainment source is comprised of multiple clips of other entertainment sources.

32. A system for generating a workout profile that is based on and synchronized with an entertainment source, the system comprising:
a processor; and
a computer readable medium storing computer-readable instructions which, when executed by the processor, cause the processor to:
identify non-quantitative elements in the entertainment source;
receive a biometric response to the entertainment source; and
tag each moment in the workout profile that corresponds to one of the non-quantitative elements with a setting for an exercise machine, each setting based on the biometric response.

33. The system of claim 32 comprising a user interface configured to accept manual input to a software application to identify said non-quantitative elements and tag said moments.

34. The system of claim 32 comprising a biometric sensor configured to measure the biometric response, the biometric response being of a person who is watching or listening to the entertainment source, wherein the computer-readable instructions further cause the processor to use the biometric response to identify the non-quantitative elements, tag the moments, or both identify the non-quantitative elements and tag the moments.

35. A non-transitory computer-readable medium storing computer-readable instructions which, when executed by a processor, cause the processor to generate a workout profile that is based on and synchronized with an entertainment source by:
identifying non-quantitative elements in the entertainment source;
determining a biometric response to the entertainment source; and
tagging each moment in the workout profile that corresponds to one of the non-quantitative elements with a setting for an exercise machine, each setting based on the biometric response.
